(19) United States
(12) Patent Application Publication
(10) Pub. No.: US 2016/0144083 A1
(43) Pub. Date: May 26, 2016

(54) NASAL ASPIRATOR DEVICE

(71) Applicant: Dai Vinh, Rutland, MA (US)

(72) Inventor: Dai Vinh, Rutland, MA (US)

(21) Appl. No.: 14/551,721

(22) Filed: Nov. 24, 2014

(51) Int. Cl. A61M 1/00

(52) U.S. Cl. CPC A61M 1/0072; A61M 1/0011; A61M 2205/075; A61M 2210/0618; A61B 2019/306

(57) ABSTRACT

A nasal aspirator device adjustably limits distance of insertion into a nasal cavity. The device includes a resiliently compressible bulb having an interior space. A first end of a tube is coupled to the bulb and a second end of the tube is positioned in spaced relationship to the bulb. The tube is in fluid communication with the interior space of the bulb. A ridge is coupled to and extends around an outer surface of the tube. The tube is insertable through an aperture in a guard such that the ridge abuts the guard wherein the guard is inhibited from sliding past the ridge towards the bulb.

### BACKGROUND OF THE DISCLOSURE

### Field of the Disclosure

The disclosure relates to aspirator devices and more particularly pertains to a new aspirator device for adjustably limiting distance of insertion into a nasal cavity.

### SUMMARY OF THE DISCLOSURE

An embodiment of the disclosure meets the needs presented above by generally comprising a resiliently compressible bulb having an interior space. A first end of a tube is coupled to the bulb and a second end of the tube is positioned in spaced relationship to the bulb. The tube is in fluid communication with the interior space of the bulb. A ridge is coupled to and extends around an outer surface of the tube. The tube is insertable through an aperture in a guard such that the ridge abuts the guard wherein the guard is inhibited from sliding past the ridge towards the bulb.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a top front side perspective view of a nasal aspirator device according to an embodiment of the disclosure.

FIG. 2 is a side view of an embodiment of the disclosure.

FIG. 3 is an exploded front view of an embodiment of the disclosure.

FIG. 4 is a top view of an embodiment of the disclosure.

FIG. 5 is a cross-sectional view of an embodiment of the disclosure taken along line 5-5 of FIG. 4.

FIG. 6 is a side view of an embodiment of the disclosure in use.

### DESCRIPTION OF THE PREFERRED EMBODIMENT

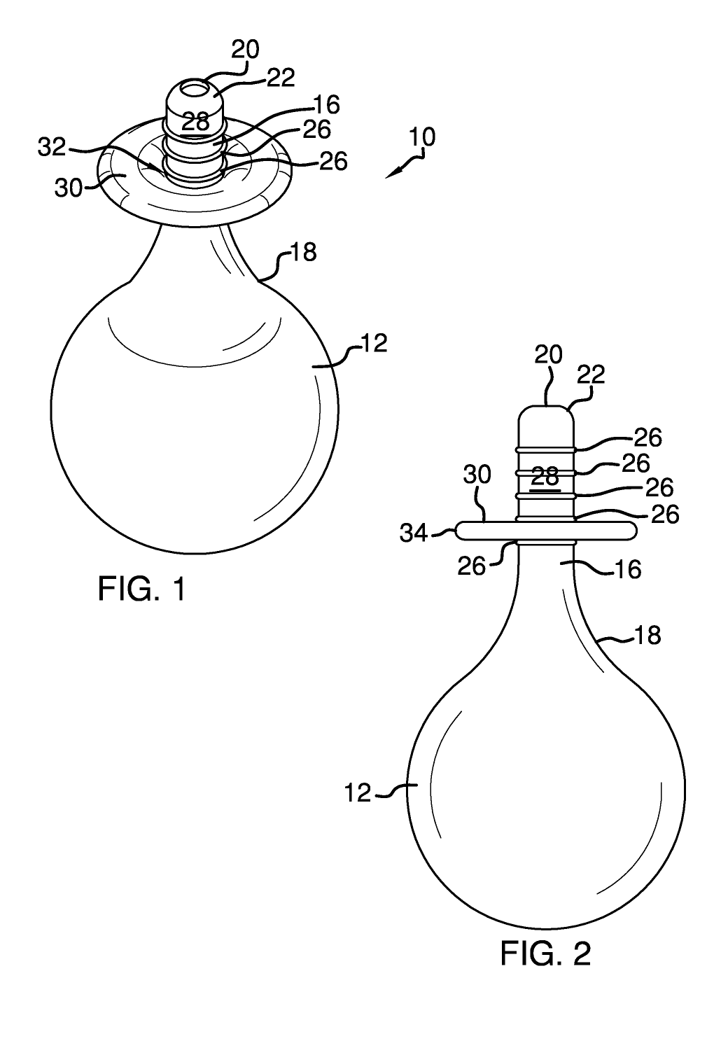

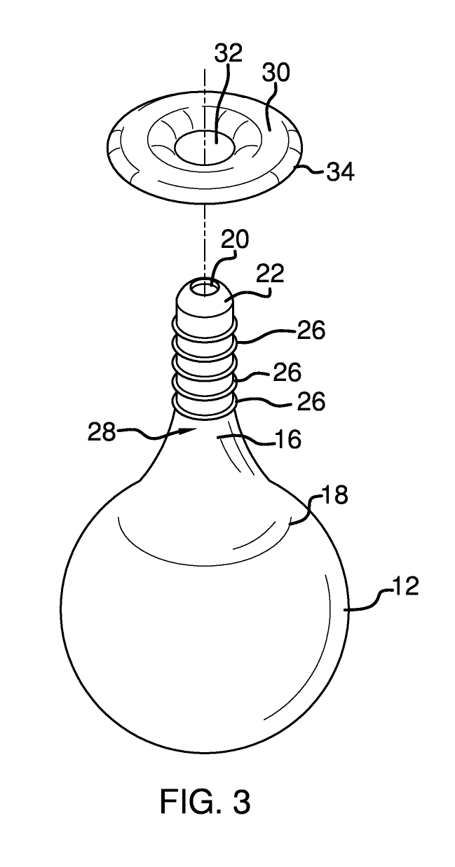

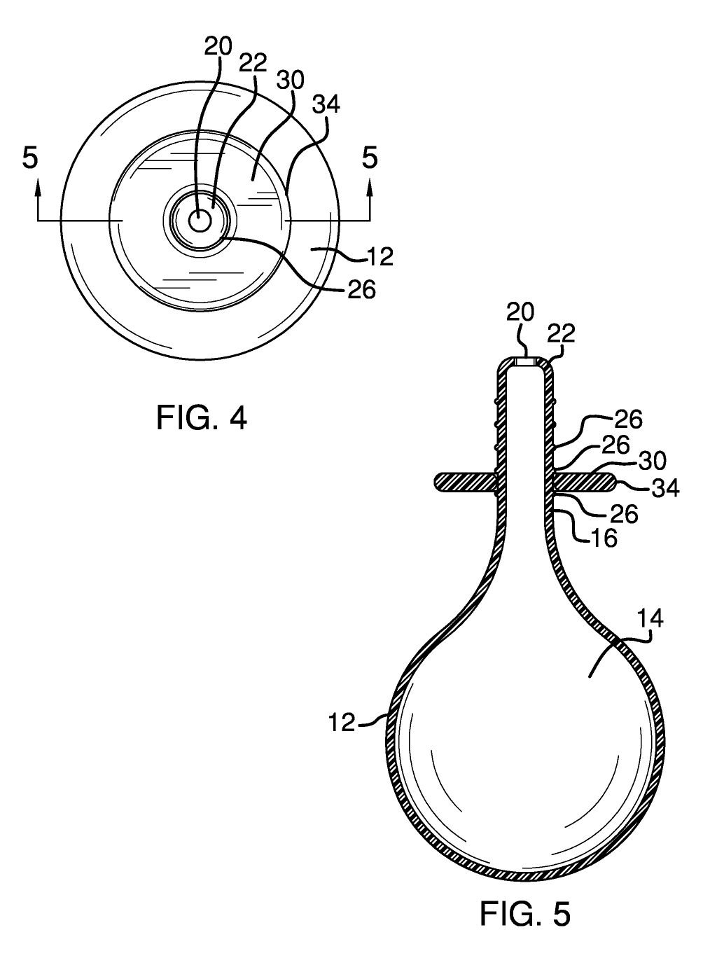

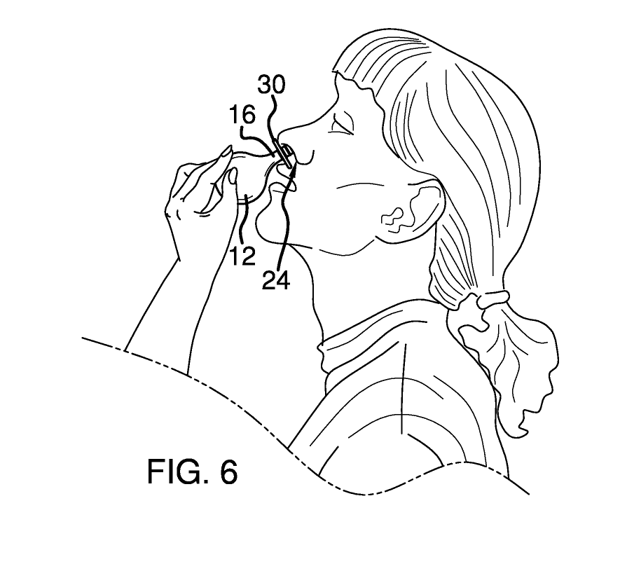

With reference now to the drawings, and in particular to FIGS. 1 through 6 thereof, a new aspirator device embodying the principles and concepts of an embodiment of the disclosure and generally designated by the reference numeral 10 will be described.

As best illustrated in FIGS. 1 through 6, the nasal aspirator device 10 generally comprises a bulb 12 having an interior space 14. The bulb 12 is constructed of rubber or the like and is resiliently compressible. A tube 16 has a first end 18 coupled to the bulb 12 and a second end 20 positioned in spaced relationship to the bulb 12. The tube 16 is in fluid communication with the interior space 14 of the bulb 12 wherein resilient expansion of the bulb 12 provides suction through the second end 20 of the tube 16 into the interior space 14. The second end 20 of the tube 16 has a rounded outer peripheral edge 22 to enhance comfort when the second end 20 of the tube 16 is inserted into a nasal cavity 24. Each of a plurality of ridges 26 is coupled to and extends around an outer surface 28 of the tube 16. The outer surface 28 of the tube 16 is cylindrical adjacent to the second end 20 of the tube 16. The ridges 26 are spaced apart along the outer surface 28 of the tube 16. The ridges 26 may be evenly spaced along the outer surface 28 of the tube 16 and each ridge 26 is oriented transverse to a longitudinal axis of the tube 16.

A guard 30 has an aperture 32. The aperture 32 may be centrally located extending through the guard 30. The tube 16 is insertable through the aperture 32. The tube 16 is resiliently flexible wherein the guard 30 is positionable adjacent to a selectable one of the ridges 26 such that a distance between the second end 20 of the tube 16 and the guard 30 is adjustable and the selectable one of the ridges 26 abuts the guard 30 wherein the guard 30 is inhibited from sliding past the selectable one of the ridges 26 towards the bulb 12. Spacing between the ridges 26 is complementary to a thickness of the guard 30 around the aperture 32 such that the guard 30 is positionable between adjacently positioned ridges 26 wherein the adjacently positioned ridges 26 inhibit sliding of the guard 30 on the tube 16 both towards and away from the second end 20 of the tube 16. The guard 30 may be disc-shaped. The guard 30 has a rounded outer perimeter edge 34.

In use, the guard 30 is positioned on the tube 16 adjacent to a ridge 26 or between two of the ridges 26 to set the guard 30 at a desired distance from the second end 20 of the tube 16. The second end 20 of the tube 16 is inserted into the nasal cavity 24 and the guard 30 prevents penetration of the second end 20 beyond the distance between the guard 30 and the second end 20 of the tube 16. The bulb 12 is manipulated to provide suction through the second end 20 of the tube 16 while the second end 20 of the tube 16 is positioned in the nasal cavity 24.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A nasal aspirator device comprising:
a bulb having an interior space, said bulb being resiliently compressible;
a tube, said tube having a first end coupled to said bulb and a second end positioned in spaced relationship to said bulb, said tube being in fluid communication with said interior space of said bulb wherein resilient expansion of said bulb provides suction through said second end of said tube;
a ridge coupled to and extending around an outer surface of said tube; and
a guard, said guard having an aperture, said tube being insertable through said aperture such that said ridge abuts said guard wherein said guard is inhibited from sliding past said ridge towards said bulb.

2. The device of claim 1, further comprising said ridge being one of a plurality of ridges coupled to said outer surface of said tube, said ridges being spaced apart along said outer surface of said tube, said tube being resiliently flexible wherein said guard is positionable adjacent to a selectable one of said ridges such that a distance between said second end of said tube and said guard is adjustable.

3. The device of claim 2, further comprising said ridges being evenly spaced along said outer surface of said tube.

4. The device of claim 3, further comprising spacing between said ridges being complementary to a thickness of said guard around said aperture such that said guard is positionable between adjacently positioned said ridges wherein said adjacently positioned ridges inhibit sliding of said guard on said tube both towards and away from said second end of said tube.

5. The device of claim 2, further comprising each said ridge being oriented transverse to a longitudinal axis of said tube.

6. The device of claim 1, further comprising said outer surface of said tube being cylindrical.

7. The device of claim 1, further comprising said guard being disc-shaped.

8. The device of claim 7, further comprising said guard having a rounded outer perimeter edge.

9. The device of claim 1, further comprising said second end of said tube having a rounded outer peripheral edge.

10. A nasal aspirator device comprising:
a bulb having an interior space, said bulb being resiliently compressible;
a tube, said tube having a first end coupled to said bulb and a second end positioned in spaced relationship to said bulb, said tube being in fluid communication with said interior space of said bulb wherein resilient expansion of said bulb provides suction through said second end of said tube, said second end of said tube having a rounded outer peripheral edge;
a plurality of ridges, each said ridge being coupled to and extending around an outer surface of said tube, said outer surface of said tube being cylindrical, said ridges being spaced apart along said outer surface of said tube, said ridges being evenly spaced along said outer surface of said tube, each said ridge being oriented transverse to a longitudinal axis of said tube; and
a guard, said guard having an aperture, said tube being insertable through said aperture, said tube being resiliently flexible wherein said guard is positionable adjacent to a selectable one of said ridges such that a distance between said second end of said tube and said guard is adjustable and said selectable one of said ridges abuts said guard wherein said guard is inhibited from sliding past said selectable one of said ridges towards said bulb, spacing between said ridges being complementary to a thickness of said guard around said aperture such that said guard is positionable between adjacently positioned said ridges wherein said adjacently positioned ridges inhibit sliding of said guard on said tube both towards and away from said second end of said tube, said guard being disc-shaped, said guard having a rounded outer perimeter edge.
